(19) United States
(12) Patent Application Publication
(10) Pub. No.: US 2003/0224954 A1
(43) Pub. Date: Dec. 4, 2003

(54) Conditioning shampoo containing aminosilicone

(75) Inventors: Robert Lee Wells, Cincinnati, OH (US); Kendrick Jon Hughes, Cincinnati, OH (US)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Proctor & Gamble Company

(21) Appl. No.: 10/454,564

(22) Filed: Jun. 4, 2003

Related U.S. Application Data
(60) Provisional application No. 60/385,798, filed on Jun. 4, 2002.
(60) Provisional application No. 60/423,632, filed on Nov. 4, 2002.

(51) Int. Cl. A61K 7/075

(52) U.S. Cl. 510/119; 510/122; 510/124; 510/499

(57) ABSTRACT

Disclosed is are hair conditioning shampoo compositions comprising a) a detersive surfactant, b) an aminosilicone having a viscosity of from about 1,000 cs to about 1,000,000 cs, and less than about 0.5% nitrogen by weight of the aminosilicone and, c) an aqueous carrier. Also disclosed are hair conditioning shampoo compositions comprising a) a detersive surfactant, b) an aminosilicone having less than about 0.5% nitrogen by weight of the aminosilicone, c) a non-amino-functionalized silicone having a viscosity of at least about 10,000 cs and, d) an aqueous carrier.

### CROSS REFERENCE TO RELATED APPLICATION

The application claims the benefit of U.S. Provisional application Serial No. 60/385,798 (Case 8957P), filed on Jun. 4, 2002, and U.S. Provisional application Serial No. 60/423,632 (Case 8957P2), filed on Nov. 4, 2002.

### FIELD

The present invention relates to conditioning shampoo compositions containing select combinations of hair conditioning agents, which provide improved hair conditioning performance.

### BACKGROUND

Conditioning shampoos containing various combinations of detersive surfactant and hair conditioning agents are known. These shampoos have become more popular among consumers as a means of conveniently obtaining hair conditioning and hair cleansing performance all from a single hair care product.

One approach at improving the overall conditioning performance from a conditioning shampoo involves the use of silicone conditioning agents. These conditioners provide improved hair conditioning performance, and in particular improve the softness and clean feel of dry conditioned hair. These silicone conditioners, however, provide less than optimal deposition of the silicone component to the hair and/or less than optimum conditioning benefits such as dry hair smoothness, hair strand alignment (e.g., minimize frizziness), and ease of combing

Based on the foregoing, there is a need for a conditioning shampoo composition, which provides improved deposition of the silicone component and/or improved hair conditioning benefits.

### SUMMARY

One embodiment of the present invention is directed to a) a conditioning shampoo composition comprising a) a detersive surfactant, b) an aminosilicone having a viscosity of from about 1,000 cs to about 1,000,000 cs, and less than about 0.5% nitrogen by weight of the aminosilicone and, c) an aqueous carrier.

In another embodiment, the conditioning shampoo composition comprises a) a detersive surfactant, b) an aminosilicone having less than about 0.5% nitrogen by weight of the aminosilicone, c) a non-amino-functionalized silicone having a viscosity of at least about 10,000 cs and, d) an aqueous carrier. These and other aspects of the present invention are discussed in more detail, below.

Another embodiment of the present invention relates to a method of making a conditioning shampoo composition comprising mixing together: a) a previously formed blend of aminosilicone and non-amino-functionalized silicone, wherein the aminosilicone has less than about 0.5% nitrogen by weight of the aminosilicone and the non-amino-functionalized silicone has a viscosity of at least about 10,000 cs; b) a detersive surfactant, and; c) an aqueous carrier.

These and other features, aspects, and advantages of the present invention will become evident to those skilled in the art from a reading of the present disclosure.

### BRIEF DESCRIPTION OF THE DRAWINGS

While the specification concludes with claims that particularly point out and distinctly claim the invention, it is believed the present invention will be better understood from the following description of preferred embodiments taken in conjunction with the accompanying drawings in which:

Table 1 demonstrates the relationship between friction and % Nitrogen for silicone treated hair for amino functionalized silicone.

### DETAILED DESCRIPTION

All documents cited are, in relevant part, incorporated herein by reference. The citation of any document is not to be construed as an admission that it is prior art with respect to the present invention.

All percentages are by weight of total composition unless specifically stated otherwise.

All ratios are weight ratios unless specifically stated otherwise.

Except as otherwise noted, all amounts including quantities, percentages, portions, and proportions, are understood to be modified by the word “about”, and amounts are not intended to indicate significant digits.

Except as otherwise noted, the articles “a”, “an”, and “the” mean “one or more”

Herein, “μ” means microns.

Herein, “comprising” means that other steps and other ingredients which do not affect the end result can be added. This term encompasses the terms “consisting of” and “consisting essentially of”. The compositions and methods/processes of the present invention can comprise, consist of, and consist essentially of the essential elements and limitations of the invention described herein, as well as any of the additional or optional ingredients, components, steps, or limitations described herein.

Herein, “cs” means centistoke.

Herein, “molecular weight” means the weight average” MW, and can be measured by gel permeation chromotography (GPC).

Herein, “PDMS” means polydimethylsiloxane.

Herein, “graft” means attached to a backbone at any position other than an end group.

Herein, “terminal” means attached to a backbone at an end group.

The aspects and embodiments of the present invention set forth in this document have many advantages. For example, applicants have discovered that aminosilicones at a certain viscosity range, when used in a conditioning shampoo composition, provide a surprisingly improved level of deposition of the aminosilicone. Various embodiments of the present invention further address the need for providing surprisingly improved hair conditioning benefits, including, e.g., dry hair softness, smoothness, hair strand alignment (i.e., minimization of frizzy hair), ease of dry combing and/or a general conditioned hair feel.

I. Detersive Surfactant

The hair conditioning shampoo composition of the present invention includes a detersive surfactant. The detersive surfactant component is included to provide cleaning performance to the composition. The detersive surfactant component in turn includes anionic detersive surfactant, zwitterionic or amphoteric detersive surfactant, or a combination thereof. Such surfactants should be physically and chemically compatible with the essential components described herein, or should not otherwise unduly impair product stability, aesthetics or performance.

Suitable anionic detersive surfactant components for use in the hair conditioning shampoo composition include those which are known for use in hair care or other personal care cleansing compositions. The concentration of the anionic surfactant component in the composition should be sufficient to provide the desired cleaning and lather performance, and generally range from about 5% to about 50%, preferably from about 8% to about 30%, more preferably from about 10% to about 25%, even more preferably from about 12% to about 22%.

Preferred anionic surfactants suitable for use in the compositions are the alkyl and alkyl ether sulfates. These materials have the respective formula ROSO₃M and RO(C₂H₄O)_{X}SO₃M, wherein R is alkyl or alkenyl of from about 8 to about 18 carbon atoms, x is an integer having a value of from 1 to 10, and M is a cation such as ammonium, alkanolamines, such as triethanolamine, monovalent metals, such as sodium and potassium, and polyvalent metal cations, such as magnesium, and calcium.

Preferably, R has from about 8 to about 18 carbon atoms, more preferably from about 10 to about 16 carbon atoms, even more preferably from about 12 to about 14 carbon atoms, in both the alkyl and alkyl ether sulfates. The alkyl ether sulfates are typically made as condensation products of ethylene oxide and monohydric alcohols having from about 8 to about 24 carbon atoms. The alcohols can be synthetic or they can be derived from fats, e.g., coconut oil, palm kernel oil, and tallow. Lauryl alcohol and straight chain alcohols derived from coconut oil or palm kernel oil are preferred. Such alcohols are reacted with between about 0 and about 10, preferably from about 2 to about 5, more preferably about 3, molar proportions of ethylene oxide, and the resulting mixture of molecular species having, for example, an average of 3 moles of ethylene oxide per mole of alcohol, is sulfated and neutralized.

Other suitable anionic detersive surfactants are the water-soluble salts of organic, sulfuric acid reaction products conforming to the formula [R₁—SO₃-M] where R¹ is a straight or branched chain, saturated, aliphatic hydrocarbon radical having from about 8 to about 24, preferably about 10 to about 18, carbon atoms; and M is a cation described hereinbefore.

Still other suitable anionic detersive surfactants are the reaction products of fatty acids esterified with isethionic acid and neutralized with sodium hydroxide where, for example, the fatty acids are derived from coconut oil or palm kernel oil; sodium or potassium salts of fatty acid amides of methyl tauride in which the fatty acids, for example, are derived from coconut oil or palm kernel oil. Other similar anionic surfactants useful in the subject compositions are described in U.S. Pat. Nos. 2,486,921; 2,486,922; and 2,396,278.

Other anionic detersive surfactants suitable for use in the compositions are the succinnates, examples of which include disodium N-octadecylsulfosuccinnate; disodium lauryl sulfosuccinate; diammonium lauryl sulfosuccinate; tetrasodium N-(1,2-dicarboxyethyl)-N-octadecylsulfosuccinnate; diamyl ester of sodium sulfosuccinic acid; dihexyl ester of sodium sulfosuccinic acid; and dioctyl esters of sodium sulfosuccinic acid.

Other suitable anionic detersive surfactants include olefin sulfonates having about 10 to about 24 carbon atoms. In addition to the true alkene sulfonates and a proportion of hydroxy-alkanesulfonates, the olefin sulfonates can contain minor amounts of other materials, such as alkene disulfonates depending upon the reaction conditions, proportion of reactants, the nature of the starting olefins and impurities in the olefin stock and side reactions during the sulfonation process. A non-limiting example of such an alpha-olefin sulfonate mixture is described in U.S. Pat. No. 3,332,880.

Another class of anionic detersive surfactants suitable for use in the compositions are the beta-alkyloxy alkane sulfonates. These surfactants conform to the formula I:

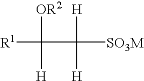

where R¹ is a straight chain alkyl group having from about 6 to about 20 carbon atoms, R² is a lower alkyl group having from about 1 to about 3 carbon atoms, preferably 1 carbon atom, and M is a water-soluble cation as described hereinbefore.

Preferred anionic detersive surfactants for use in the compositions include ammonium lauryl sulfate, ammonium laureth sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, sodium laureth sulfate, potassium lauryl sulfate, potassium laureth sulfate, sodium lauryl sarcosinate, sodium lauroyl sarcosinate, lauryl sarcosine, cocoyl sarcosine, ammonium cocoyl sulfate, ammonium lauroyl sulfate, sodium cocoyl sulfate, sodium lauroyl sulfate, potassium cocoyl sulfate, potassium lauryl sulfate, triethanolamine lauryl sulfate, triethanolamine lauryl sulfate, monoethanolamine cocoyl sulfate, monoethanolamine lauryl sulfate, sodium tridecyl benzene sulfonate, sodium dodecyl benzene sulfonate, sodium cocoyl isethionate and combinations thereof.

Suitable amphoteric or zwitterionic detersive surfactants for use in the composition herein include those which are known for use in hair care or other personal care cleansing. Concentration of such amphoteric detersive surfactants preferably ranges from about 0.5% to about 20%, preferably from about 1% to about 10%. Non-limiting examples of suitable zwitterionic or amphoteric surfactants are described in U.S. Pat. No. 5,104,646 (Bolich Jr. et al.), U.S. Pat. No. 5,106,609 (Bolich Jr. et al.).

Amphoteric detersive surfactants suitable for use in the composition are well known in the art, and include those surfactants broadly described as derivatives of aliphatic secondary and tertiary amines in which the aliphatic radical can be straight or branched chain and wherein one of the aliphatic substituents contains from about 8 to about 18 carbon atoms and one contains an anionic group such as carboxy, sulfonate, sulfate, phosphate, or phosphonate. Preferred amphoteric detersive surfactants for use in the present invention include cocoamphoacetate, cocoamphodiacetate, lauroamphoacetate, lauroamphodiacetate, and mixtures thereof.

Zwitterionic detersive surfactants suitable for use in the compositions are well known in the art, and include those surfactants broadly described as derivatives of aliphatic quaternary ammonium, phosphonium, and sulfonium compounds, in which the aliphatic radicals can be straight or branched chain, and wherein one of the aliphatic substituents contains from about 8 to about 18 carbon atoms and one contains an anionic group such as carboxy, sulfonate, sulfate, phosphate or phosphonate. Zwitterionics such as betaines are preferred.

The compositions of the present invention may further comprise additional surfactants for use in combination with the anionic detersive surfactant component described hereinbefore. Suitable optional surfactants include nonionic and cationic surfactants. Any such surfactant known in the art for use in hair or personal care products may be used, provided that the optional additional surfactant is also chemically and physically compatible with the essential components of the composition, or does not otherwise unduly impair product performance, aesthetics or stability. The concentration of the optional additional surfactants in the composition may vary with the cleansing or lather performance desired, the optional surfactant selected, the desired product concentration, the presence of other components in the composition, and other factors well known in the art.

Non-limiting examples of other anionic, zwitterionic, amphoteric or optional additional surfactants suitable for use in the compositions are described in McCutcheon's, Emulsifiers and Detergents, 1989 Annual, published by M. C. Publishing Co., and U.S. Pat. Nos. 3,929,678, 2,658,072; 2,438,091; 2,528,378.

II. Silicone

The hair conditioning shampoo composition of the present invention further includes a silicone. In one embodiment, the silicone component is made up of an amino functionalized silicone (“aminosilicone”). In another embodiment, the silicone component is a combination of aminosilicone and non-amino-functionalized silicone (i.e., a silicone which contains no amine functional groups; herein “NAFS”). In such an embodiment, the aminosilicone and NAFS preferably form emulsion drops or particles containing a blend of aminosilicone and NAFS.

Preferably the silicones used in the present invention have a particle size of less than about 50 μ. Embodiments having a silicone particle size of less than about 5 μ preferably further include a deposition aid. Examples of preferred deposition aids are discussed in more detail, below. Hair shampoo conditioning composition embodiments employing silicones having a particle size of from about 5 μ to about 50 μ preferably do not include a depostion aid.

Particle size may be measured by means of a laser light scattering technique, using a Horiba model LA-910 Laser Scattering Particle Size Distribution Analyzer (Horiba Instruments, Inc.; Irivine, Calif., USA).

The viscosity of silicones discussed herein is measured at 25° C.

A. Aminosilicone

Herein “aminosilicone” means any amine functionalized silicone; i.e., a silicone containing at least one primary amine, secondary amine, tertiary amine, or a quaternary ammonium group. Preferred aminosilicones will typically have less than about 0.5% nitrogen by weight of the aminosilicone, more preferably less than about 0.2%, more preferably still, less than about 0.10%. Higher levels of nitrogen (amine functional groups) in the aminosilicone tend to result in both less friction reduction and very low deposition of the aminosilicone to the hair; and consequently, minimal to no conditioning benefit from the aminosilicone component.

In a preferred embodiment, the aminosilicone has a viscosity of from about 1,000 cs to about 1,000,000 cs, more preferably 2,000 cs to 600,000 cs, more preferably from about 4,000 cs to about 400,000 cs. The viscosity of the aminosilicone is more critical in embodiments which contain the aminosilicone as the only silicone component. However, in embodiments that contain aminosilicone in combination with a NAFS, the viscosity of the aminosilicone component becomes less critical when the aminosilicone makes up the minority of the total silicone in such a multi-silicone containing embodiment. Aminosilicones may be graft or terminal. Preferred graft aminosilicones have viscosities of from about 1,000 to about 50,000 cs, more preferably from about 5,000 to about 30,000 cs, still more preferably from about 10,000 to about 25,000 cs. Preferred terminal aminosilicones have viscosities of from about 1,000 to about 1,000,000 cs, more preferably from about 50,000 to about 500,000 cs, still more preferably from about 100,000 to about 300,000 cs.

Example preferred aminosilicones for use in embodiments of the subject invention include but are not limited to, those which conform to the general formula (II):

(R₁)ₐG₃₋ₐ-Si—(—OSiG₂)ₙ—(—OSiG_{b}(R₁)_{2-b)m}-O—SiG₃₋ₐ(R₁)ₐ

wherein G is hydrogen, phenyl, hydroxy, or C₁-C₈ alkyl, preferably methyl; a is 0 or an integer having a value from 1 to 3, preferably 1; b is 0, 1 or 2, preferably 1; n is a number from 10 to 1,999, preferably from 49 to 500; m is an integer from 0 to 2,000, preferably from 0 to 10; the sum of n and m is a number from 100 to 2,000, preferably from 400 to 1800; R₁ is a monovalent radical conforming to the general formula C_{q}H_{2q}L, wherein q is an integer having a value from 2 to 8 and L is selected from the following groups: —N(R₂)CH₂—CH₂—N(R₂)₂; —N(R₂)₂; —N(R₂)₃A⁻; —N(R₂)CH2—CH₂—NR₂H₂A⁻; wherein R₂ is hydrogen, phenyl, benzyl, or a saturated hydrocarbon radical, preferably an alkyl radical from about C₁ to about C₂₀, and A⁻ is a halide ion.

A preferred aminosilicone corresponding to formula (II) has m=0, a=1, q=3, n˜1600, and L is —N(CH₃)₂

Other aminosilicone polymers which may be used in the compositions of the present invention are represented by the general formula (III):

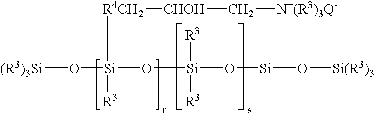

wherein R³ is a monovalent hydrocarbon radical from C₁ to C₁₈, preferably an alkyl or alkenyl radical, such as methyl; R₄ is a hydrocarbon radical, preferably a C₁ to C₁₈ alkylene radical or a C₁₀ to C₁₈ alkyleneoxy radical, more preferably a C₁ to C₈ alkyleneoxy radical; Q⁻ is a halide ion, preferably chloride; r is an average statistical value from 2 to 20, preferably from 2 to 8; s is an average statistical value from 20 to 200, preferably from 20 to 50. A preferred polymer of this class is known as UCARE SILICONE ALE 56™, available from Union Carbide.

B. Non-Amino-Functionalized Silicone (NAFS)

In embodiments containing NAFS, the weight ratio of aminosilicone to NAFS is preferably from about 1:2 to about 1:99.9, more preferably from about 1:5 to about 1:99, more preferably 5:95. Preferably the NAFS has a viscosity of at least about 10,000 cs, more preferably from about 60,000 cs to about 2,000,000 cs more preferably from about 100,000 cs to about 500,000 cs.

The NAFS component may comprise volatile NAFS, non-volatile NAFS, or combinations thereof. Preferred are non-volatile NAFS. If volatile NAFS are present, it will typically be incidental to their use as a solvent or carrier for commercially available forms of non-volatile NAFS materials ingredients, such as NAFS gums and resins. The NAFS may comprise a silicone fluid conditioning agent and may also comprise other ingredients, such as a NAFS resin to improve silicone fluid deposition efficiency or enhance glossiness of the hair.

The concentration of NAFS typically ranges from about 0.01% to about 10%, preferably from about 0.1% to about 8%, more preferably from about 0.1% to about 5%, more preferably from about 0.2% to about 3%. Non-limiting examples of suitable NAFS, and optional suspending agents for the silicone, are described in U.S. Reissue Pat. No. 34,584, U.S. Pat. No. 5,104,646, and U.S. Pat. No. 5,106,609.

Background material on silicones including sections discussing silicone fluids, gums, and resins, as well as manufacture of silicones, are found in Encyclopedia of Polymer Science and Engineering, vol. 15, 2d ed., pp 204-308, John Wiley & Sons, Inc. (1989).

Embodiments containing a blend of aminosilicone and NAFS provide several benefits, including improved deposition of the silicone component and improved hair feel over compositions containing NAFS as the sole silicone component. Additionally, as aminosilicones are generally more expensive than more NAFS, compositions containing both materials will generally be less expensive than those containing only aminosilicone as the silicone component, yet still provide improved hair conditioning versus compositions containing NAFS as the sole silicone component.

1. NAFS Oils

NAFS fluids include NAFS oils, which are flowable silicone materials having a viscosity, as measured at 25° C., less than 1,000,000 cs, preferably from about 5 cs to about 1,000,000 cs, more preferably from about 100 cs to about 600,000 cs. Suitable NAFS oils for use in the compositions of the present invention include polyalkyl siloxanes, polyaryl siloxanes, polyalkylaryl siloxanes, polyether siloxane copolymers, and mixtures thereof. Other insoluble, non-volatile NAFS fluids having hair conditioning properties may also be used.

NAFS oils include polyalkyl or polyaryl siloxanes which conform to the following Formula (IV):

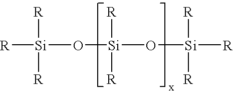

wherein R is aliphatic, preferably alkyl or alkenyl, or aryl, R can be substituted or unsubstituted, and x is an integer from 1 to about 8,000. Suitable R groups for use in the compositions of the present invention include, but are not limited to: alkoxy, aryloxy, alkylaryl, arylalkyl, arylalkenyl, alkamino, and ether-substituted, hydroxyl-substituted, and halogen-substituted aliphatic and aryl groups. Suitable R groups also include cationic amines and quaternary ammonium groups.

Preferred alkyl and alkenyl substituents are C₁ to C₅ alkyls and alkenyls, more preferably from C₁ to C₄, more preferably from C₁ to C₂. The aliphatic portions of other alkyl-, alkenyl-, or alkynyl-containing groups (such as alkoxy, alkaryl, and alkamino) can be straight or branched chains, and are preferably from C₁ to C₅, more preferably from C₁ to C₄, even more preferably from C₁ to C₃, more preferably from C₁ to C₂. As discussed above, the R substituents can also contain amino functionalities (e.g. alkylamino groups), which can be primary, secondary or tertiary amines or quaternary ammonium. These include mono-, di- and tri-alkylamino and alkoxyamino groups, wherein the aliphatic portion chain length is preferably as described herein.

2. NAFS Gums

Other NAFS fluids suitable for use in the compositions of the present invention are the insoluble NAFS gums. These gums are polyorganosiloxane materials having a viscosity, as measured at 25° C., of greater than or equal to 1,000,000 cs. NAFS gums are described in U.S. Pat. No. 4,152,416; Noll and Walter, Chemistry and Technology of Silicones, New York: Academic Press (1968); and in General Electric Silicone Rubber Product Data Sheets SE 30, SE 33, SE 54 and SE 76. Specific non-limiting examples of NAFS gums for use in the compositions of the present invention include polydimethylsiloxane, (polydimethylsiloxane) (methylvinyl-siloxane) copolymer, poly(dimethylsiloxane) (diphenyl siloxane)(methylvinylsiloxane) copolymer and mixtures thereof.

3. High Refractive Index NAFS

Other non-volatile, insoluble NAFS that are suitable for use in the compositions of the present invention are those known as “high refractive index silicones,” having a refractive index of at least about 1.46, preferably at least about 1.48, more preferably at least about 1.52, more preferably at least about 1.55. The refractive index of the polysiloxane fluid will generally be less than about 1.70, typically less than about 1.60. In this context, polysiloxane “fluid” includes oils as well as gums.

The high refractive index polysiloxane fluid includes those represented by general Formula (IV) above, as well as cyclic polysiloxanes such as those represented by Formula (V) below:

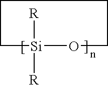

wherein R is as defined above, and n is a number from about 3 to about 7, preferably from about 3 to about 5.

The high refractive index polysiloxane fluids contain an amount of aryl-containing R substituents sufficient to increase the refractive index to the desired level, which is described herein. Additionally, R and n must be selected so that the material is non-volatile.

Aryl-containing substituents include those which contain alicyclic and heterocyclic five and six member aryl rings and those which contain fused five or six member rings. The aryl rings themselves can be substituted or unsubstituted.

Generally, the high refractive index polysiloxane fluids will have a degree of aryl-containing substituents of at least about 15%, preferably at least about 20%, more preferably at least about 25%, even more preferably at least about 35%, more preferably at least about 50%. Typically, the degree of aryl substitution will be less than about 90%, more generally less than about 85%, preferably from about 55% to about 80%.

Preferred high refractive index polysiloxane fluids have a combination of phenyl or phenyl derivative substituents (more preferably phenyl), with alkyl substituents, preferably C₁-C₄ alkyl (more preferably methyl), hydroxy, or C₁-C₄ alkylamino (preferably —R¹NHR²NH2 wherein each R¹ and R² independently is a C₁-C₃ alkyl, alkenyl, and/or alkoxy).

When high refractive index NAFS are used in the compositions of the present invention, they are preferably used in solution with a spreading agent, such as a NAFS resin or a surfactant, to reduce the surface tension by a sufficient amount to enhance spreading and thereby enhance the glossiness (subsequent to drying) of hair treated with the compositions.

NAFS fluids suitable for use in the compositions of the present invention are disclosed in U.S. Pat. No. 2,826,551, U.S. Pat. No. 3,964,500, U.S. Pat. No. 4,364,837, British Pat. No. 849,433, and Silicon Compounds, Petrarch Systems, Inc. (1984).

4. NAFS Resins

NAFS resins may be included in the compositions of the present invention. These resins are highly cross-linked polymeric siloxane systems. The cross-linking is introduced through the incorporation of trifunctional and tetrafunctional silanes with monofunctional or difunctional, or both, silanes during manufacture of the NAFS resin.

NAFS materials and NAFS resins in particular, can conveniently be identified according to a shorthand nomenclature system known to those of ordinary skill in the art as “MDTQ” nomenclature. Under this system, the NAFS is described according to presence of various siloxane monomer units which make up the NAFS. Briefly, the symbol M denotes the monofunctional unit (CH₃)₃SiO_{0.5}; D denotes the difunctional unit (CH₃)₂SiO; T denotes the trifunctional unit (CH₃)SiO_{1.5}; and Q denotes the quadra- or tetra-functional unit SiO₂. Primes of the unit symbols (e.g. M′, D′, T′, and Q′) denote substituents other than methyl, and must be specifically defined for each occurrence.

Preferred NAFS resins for use in the compositions of the present invention include, but are not limited to MQ, MT, MTQ, MDT and MDTQ resins. Methyl is a preferred NAFS substituent. Especially preferred silicone resins are MQ resins, wherein the M:Q ratio is from about 0.5:1.0 to about 1.5:1.0 and the average molecular weight of the NAFS resin is from about 1000 to about 10,000.

The weight ratio of the non-volatile NAFS fluid, having refractive index below 1.46, to the NAFS resin component, when used, is preferably from about 4:1 to about 400:1, more preferably from about 9:1 to about 200:1, more preferably from about 19:1 to about 100:1, particularly when the NAFS fluid component is a polydimethylsiloxane fluid or a mixture of polydimethylsiloxane fluid and polydimethylsiloxane gum as described herein. Insofar as the silicone resin forms a part of the same phase in the compositions hereof as the silicone fluid, i.e. the conditioning active, the sum of the fluid and resin should be included in determining the level of NAFS conditioning agent in the composition.

III. Aqueous Carrier

Preferred embodiments of the present invention are in the form of pourable liquids (under ambient conditions). Such compositions will therefore typically comprise an aqueous carrier, which is present at a level of from about 20% to about 95%, more preferably from about 60% to about 85%. The aqueous carrier may comprise water, or a miscible mixture of water and organic solvent, but preferably comprises water with minimal or no significant concentrations of organic solvent, except as otherwise incidentally incorporated into the composition as minor ingredients of other essential or optional components.

IV. Deposition Aid

In an embodiment of the present invention, the conditioning shampoo composition further includes a deposition aid. Herein, “deposition aid” means an agent which enhances deposition of the silicone component from the conditioning shampoo composition onto the intended site during use, i.e., the hair and/or scalp. Preferred embodiments include from about 0.01 to about 10% deposition aid, more preferably, more preferably from about 0.1 to about 2%.

The deposition aid is preferably a cationic polymer. Preferred hair conditioning shampoo composition embodiments preferably have from 0.05% to about 3% cationic polymer, more preferably from about 0.075% to about 2.0%, more preferably from about 0.1% to about 1.0%. Preferred cationic polymers will have cationic charge densities of at least about 0.9 meq/gm, preferably at least about 1.2 meq/gm, more preferably at least about 1.5 meq/gm, but also preferably less than about 7 meq/gm, more preferably less than about 5 meq/gm, at the pH of intended use of the composition, which pH will generally range from about pH 3 to about pH 9, preferably between about pH 4 and about pH 8. Herein, “cationic charge density” of a polymer refers to the ratio of the number of positive charges on the polymer to the molecular weight of the polymer. The average molecular weight of such suitable cationic polymers will generally be between about 10,000 and 10 million, preferably between about 50,000 and about 5 million, more preferably between about 100,000 and about 3 million.

Suitable cationic polymers for use in the compositions of the present invention contain cationic nitrogen-containing moieties such as quaternary ammonium or cationic protonated amino moieties. The cationic protonated amines can be primary, secondary, or tertiary amines (preferably secondary or tertiary), depending upon the particular species and the selected pH of the composition. Any anionic counterions can be used in association with the cationic polymers so long as the polymers remain soluble in water, in the composition, or in a coacervate phase of the composition, and so long as the counterions are physically and chemically compatible with the essential components of the composition or do not otherwise unduly impair product performance, stability or aesthetics. Non-limiting examples of such counterions include halides (e.g., chloride, fluoride, bromide, iodide), sulfate and methylsulfate.

Non-limiting examples of such polymers are described in the CTFA Cosmetic Ingredient Dictionary, 3rd edition, edited by Estrin, Crosley, and Haynes, (The Cosmetic, Toiletry, and Fragrance Association, Inc., Washington, D.C. (1982)).

Non-limiting examples of suitable cationic polymers include copolymers of vinyl monomers having cationic protonated amine or quaternary ammonium functionalities with water soluble spacer monomers such as acrylamide, methacrylamide, alkyl and dialkyl acrylamides, alkyl and dialkyl methacrylamides, alkyl acrylate, alkyl methacrylate, vinyl caprolactone or vinyl pyrrolidone.

Suitable cationic protonated amino and quaternary ammonium monomers, for inclusion in the cationic polymers of the composition herein, include vinyl compounds substituted with dialkylaminoalkyl acrylate, dialkylaminoalkyl methacrylate, monoalkylaminoalkyl acrylate, monoalkylaminoalkyl methacrylate, trialkyl methacryloxyalkyl ammonium salt, trialkyl acryloxyalkyl ammonium salt, diallyl quaternary ammonium salts, and vinyl quaternary ammonium monomers having cyclic cationic nitrogen-containing rings such as pyridinium, imidazolium, and quaternized pyrrolidone, e.g., alkyl vinyl imidazolium, alkyl vinyl pyridinium, alkyl vinyl pyrrolidone salts.

Other suitable cationic polymers for use in the compositions include copolymers of 1-vinyl-2-pyrrolidone and 1-vinyl-3-methylimidazolium salt (e.g., chloride salt) (referred to in the industry by the Cosmetic, Toiletry, and Fragrance Association, “CTFA”, as Polyquaternium-16); copolymers of 1-vinyl-2-pyrrolidone and dimethylaminoethyl methacrylate (referred to in the industry by CTFA as Polyquaternium-II); cationic diallyl quaternary ammonium-containing polymers, including, for example, dimethyldiallylammonium chloride homopolymer, copolymers of acrylamide and dimethyldiallylammonium chloride (referred to in the industry by CTFA as Polyquatemium 6 and Polyquaternium 7, respectively); amphoteric copolymers of acrylic acid including copolymers of acrylic acid and dimethyldiallylammonium chloride (referred to in the industry by CTFA as Polyquaternium 22), terpolymers of acrylic acid with dimethyldiallylammonium chloride and acrylamide (referred to in the industry by CTFA as Polyquaternium 39), and terpolymers of acrylic acid with methacrylamidopropyl trimethylammonium chloride and methylacrylate (referred to in the industry by CTFA as Polyquaternium 47). Preferred cationic substituted monomers are the cationic substituted dialkylaminoalkyl acrylamides, dialkylaminoalkyl methacrylamides, and combinations thereof. These preferred monomers conform the to the formula VI:

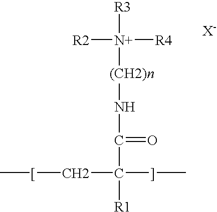

wherein R¹ is hydrogen, methyl or ethyl; each of R², R³ and R⁴ are independently hydrogen or a short chain alkyl having from about 1 to about 8 carbon atoms, preferably from about 1 to about 5 carbon atoms, more preferably from about 1 to about 2 carbon atoms; n is an integer having a value of from about 1 to about 8, preferably from about 1 to about 4; and X is a counterion. The nitrogen attached to R², R³ and R⁴ may be a protonated amine (primary, secondary or tertiary), but is preferably a quaternary ammonium wherein each of R², R³ and R⁴ are alkyl groups a non limiting example of which is polymethyacrylamidopropyl trimonium chloride, available under the trade name Polycare 133, from Rhone-Poulenc, Cranberry, N.J., U.S.A. Also preferred are copolymers of this cationic monomer with nonionic monomers such that the cationic charge density of the copolymer remains in the range specified above.

Other suitable cationic polymers for use in the composition include polysaccharide polymers, such as cationic cellulose derivatives and cationic starch derivatives. Suitable cationic polysaccharide polymers include those which conform to the formula VII:

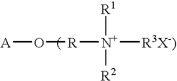

wherein A is an anhydroglucose residual group, such as a starch or cellulose anhydroglucose residual; R is an alkylene oxyalkylene, polyoxyalkylene, or hydroxyalkylene group, or combination thereof; R¹, R², and R³ independently are alkyl, aryl, alkylaryl, arylalkyl, alkoxyalkyl, or alkoxyaryl groups, each group containing up to about 18 carbon atoms, and the total number of carbon atoms for each cationic moiety (i.e., the sum of carbon atoms in R¹, R² and R³) preferably being about 20 or less; and X is an anionic counterion as described in hereinbefore.

Preferred cationic cellulose polymers are salts of hydroxyethyl cellulose reacted with trimethyl ammonium substituted epoxide, referred to in the industry (CTFA) as Polyquaternium 10 and available from Amerchol Corp. (Edison, N.J., USA) in their Polymer LR, JR, and KG series of polymers. Other suitable types of cationic cellulose includes the polymeric quaternary ammonium salts of hydroxyethyl cellulose reacted with lauryl dimethyl ammonium-substituted epoxide referred to in the industry (CTFA) as Polyquaternium 24. These materials are available from Amerchol Corp. under the tradename Polymer LM-200.

Other suitable cationic polymers include cationic guar gum derivatives, such as guar hydroxypropyltrimonium chloride, specific examples of which include the Jaguar series commercially available from Rhone-Poulenc Incorporated and the N-Hance series commercially available from Aqualon Division of Hercules, Inc. Other suitable cationic polymers include quaternary nitrogen-containing cellulose ethers, some examples of which are described in U.S. Pat. No. 3,962,418. Other suitable cationic polymers include copolymers of etherified cellulose, guar and starch, some examples of which are described in U.S. Pat. No. 3,958,581. When used, the cationic polymers herein are either soluble in the composition or are soluble in a complex coacervate phase in the composition formed by the cationic polymer and the anionic, amphoteric and/or zwitterionic detersive surfactant component described hereinbefore. Complex coacervates of the cationic polymer can also be formed with other charged materials in the composition.

Techniques for analysis of formation of complex coacervates are known in the art. For example, microscopic analyses of the compositions, at any chosen stage of dilution, can be utilized to identify whether a coacervate phase has formed. Such coacervate phase will be identifiable as an additional emulsified phase in the composition. The use of dyes can aid in distinguishing the coacervate phase from other insoluble phases dispersed in the composition.

V. Other Ingredients

Certain embodiments of the hair conditioning shampoo composition of the present invention may further include one or more optional components known for use in hair care or personal care products, provided that the optional components are physically and chemically compatible with the essential components described herein, or do not otherwise unduly impair product stability, aesthetics or performance. Individual concentrations of such optional components may range from about 0.001% to about 10%.

Non-limiting examples of optional components for use in the composition include dispersed particles, cationic polymers, other conditioning agents (hydrocarbon oils, fatty esters, other silicones), anti dandruff agents, suspending agents, viscosity modifiers, dyes, nonvolatile solvents or diluents (water soluble and insoluble), pearlescent aids, foam boosters, additional surfactants or nonionic cosurfactants, pediculocides, pH adjusting agents, perfumes, preservatives, chelants, proteins, skin active agents, sunscreens, UV absorbers, and vitamins.

A. Dispersed Particles

The compositions of the present invention may include dispersed particles. In the compositions of the present invention, it is preferable to incorporate at least 0.025% by weight of the dispersed particles, more preferably at least 0.05%, still more preferably at least 0.1%, even more preferably at least 0.25%, and yet more preferably at least 0.5% by weight of the dispersed particles. In the compositions of the present invention, it is preferable to incorporate no more than about 20% by weight of the dispersed particles, more preferably no more than about 10%, still more preferably no more than 5%, even more preferably no more than 3%, and yet more preferably no more than 2% by weight of the dispersed particles.

B. Nonionic Polymers

Polyalkylene glycols having a molecular weight of more than about 1000 are useful herein. Useful are those having the following general formula VIII:

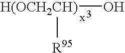

wherein R⁹⁵ is selected from the group consisting of H, methyl, and mixtures thereof. Polyethylene glycol polymers useful herein are PEG-2M (also known as Polyox WSR® N-10, which is available from Union Carbide and as PEG-2,000); PEG-SM (also known as Polyox WSR® N-35 and Polyox WSR® N-80, available from Union Carbide and as PEG-5,000 and Polyethylene Glycol 300,000); PEG-7M (also known as Polyox WSR® N-750 available from Union Carbide); PEG-9M (also known as Polyox WSR® N-3333 available from Union Carbide); and PEG-14 M (also known as Polyox WSR® N-3000 available from Union Carbide).

C. Other Conditioning Agents

Conditioning agents include any material which is used to give a particular conditioning benefit to hair and/or skin. In hair treatment compositions, suitable conditioning agents are those which deliver one or more benefits relating to shine, softness, combability, antistatic properties, wet-handling, damage, manageability, body, and greasiness. Conditioning agents (in addition to the aminosilicones and NAFS described above) useful in the compositions of the present invention typically comprise a water insoluble, water dispersible, non-volatile, liquid that forms emulsified, liquid particles. Suitable conditioning agents for use in the composition are those conditioning agents characterized generally as organic conditioning oils (e.g., hydrocarbon oils, polyolefins, and fatty esters), or those conditioning agents which otherwise form liquid, dispersed particles in the aqueous surfactant matrix herein. Such conditioning agents should be physically and chemically compatible with the essential components of the composition, and should not otherwise unduly impair product stability, aesthetics or performance.

The concentration of such other conditioning agent in the composition should be sufficient to provide the desired conditioning benefits, and as will be apparent to one of ordinary skill in the art. Such concentration can vary with the conditioning agent, the conditioning performance desired, the average size of the conditioning agent particles, the type and concentration of other components, and other like factors.

1. Organic Conditioning Oils

The conditioning component of the compositions of the present invention may also comprise from about 0.05% to about 3%, preferably from about 0.08% to about 1.5%, more preferably from about 0.1% to about 1%, of at least one organic conditioning oil as the conditioning agent, either alone or in combination with other conditioning agents, such as the silicones (described herein).

a. Hydrocarbon Oils

Suitable organic conditioning oils for use as conditioning agents in the compositions of the present invention include, but are not limited to, hydrocarbon oils having at least about 10 carbon atoms, such as cyclic hydrocarbons, straight chain aliphatic hydrocarbons (saturated or unsaturated), and branched chain aliphatic hydrocarbons (saturated or unsaturated), including polymers and mixtures thereof. Straight chain hydrocarbon oils preferably are from about C₁₂ to about C₁₉. Branched chain hydrocarbon oils, including hydrocarbon polymers, typically will contain more than 19 carbon atoms.

Specific non-limiting examples of these hydrocarbon oils include paraffin oil, mineral oil, saturated and unsaturated dodecane, saturated and unsaturated tridecane, saturated and unsaturated tetradecane, saturated and unsaturated pentadecane, saturated and unsaturated hexadecane, polybutene, polydecene, and mixtures thereof. Branched-chain isomers of these compounds, as well as of higher chain length hydrocarbons, can also be used, examples of which include highly branched, saturated or unsaturated, alkanes such as the permethyl-substituted isomers, e.g., the permethyl-substituted isomers of hexadecane and eicosane, such as 2, 2, 4, 4, 6, 6, 8, 8-dimethyl-10-methylundecane and 2, 2, 4, 4, 6, 6-dimethyl-8-methylnonane, available from Permethyl Corporation. Hydrocarbon polymers such as polybutene and polydecene. A preferred hydrocarbon polymer is polybutene, such as the copolymer of isobutylene and butene. A commercially available material of this type is L-14 polybutene from Amoco Chemical Corporation. The concentration of such hydrocarbon oils in the composition preferably range from about 0.05% to about 20%, more preferably from about 0.08% to about 1.5%, and even more preferably from about 0.1% to about 1%.

b. Polyolefins

Organic conditioning oils for use in the compositions of the present invention can also include liquid polyolefins, more preferably liquid poly-α-olefins, more preferably hydrogenated liquid poly-α-olefins. Polyolefins for use herein are prepared by polymerization of C₄ to about C₁₄ olefenic monomers, preferably from about C₆ to about C₁₂.

Non-limiting examples of olefenic monomers for use in preparing the polyolefin liquids herein include ethylene, propylene, 1-butene, 1-pentene, 1-hexene, 1-octene, 1-decene, 1-dodecene, 1-tetradecene, branched chain isomers such as 4-methyl-1-pentene, and mixtures thereof. Also suitable for preparing the polyolefin liquids are olefin-containing refinery feedstocks or effluents. Preferred hydrogenated α-olefin monomers include, but are not limited to: 1-hexene to 1-hexadecenes, 1-octene to 1-tetradecene, and mixtures thereof.

c. Fatty Esters

Other suitable organic conditioning oils for use as the conditioning agent in the compositions of the present invention include, but are not limited to, fatty esters having at least 10 carbon atoms. These fatty esters include esters with hydrocarbyl chains derived from fatty acids or alcohols (e.g. mono-esters, polyhydric alcohol esters, and di- and tri-carboxylic acid esters). The hydrocarbyl radicals of the fatty esters hereof may include or have covalently bonded thereto other compatible functionalities, such as amides and alkoxy moieties (e.g., ethoxy or ether linkages, etc.).

Specific examples of preferred fatty esters include, but are not limited to: isopropyl isostearate, hexyl laurate, isohexyl laurate, isohexyl palmitate, isopropyl palmitate, decyl oleate, isodecyl oleate, hexadecyl stearate, decyl stearate, isopropyl isostearate, dihexyldecyl adipate, lauryl lactate, myristyl lactate, cetyl lactate, oleyl stearate, oleyl oleate, oleyl myristate, lauryl acetate, cetyl propionate, and oleyl adipate.

Other fatty esters suitable for use in the compositions of the present invention are mono-carboxylic acid esters of the general formula R′COOR, wherein R′ and R are alkyl or alkenyl radicals, and the sum of carbon atoms in R′ and R is at least 10, preferably at least 22.

Still other fatty esters suitable for use in the compositions of the present invention are di- and tri-alkyl and alkenyl esters of carboxylic acids, such as esters of C₄ to C₈ dicarboxylic acids (e.g. C₁ to C₂₂ esters, preferably C₁ to C₆, of succinic acid, glutaric acid, and adipic acid). Specific non-limiting examples of di- and tri-alkyl and alkenyl esters of carboxylic acids include isocetyl stearyol stearate, diisopropyl adipate, and tristearyl citrate.

Other fatty esters suitable for use in the compositions of the present invention are those known as polyhydric alcohol esters. Such polyhydric alcohol esters include alkylene glycol esters, such as ethylene glycol mono and di-fatty acid esters, diethylene glycol mono- and di-fatty acid esters, polyethylene glycol mono- and di-fatty acid esters, propylene glycol mono- and di-fatty acid esters, polypropylene glycol monooleate, polypropylene glycol 2000 monostearate, ethoxylated propylene glycol monostearate, glyceryl mono- and di-fatty acid esters, polyglycerol poly-fatty acid esters, ethoxylated glyceryl monostearate, 1,3-butylene glycol monostearate, 1,3-butylene glycol distearate, polyoxyethylene polyol fatty acid ester, sorbitan fatty acid esters, and polyoxyethylene sorbitan fatty acid esters.

Still other fatty esters suitable for use in the compositions of the present invention are glycerides, including, but not limited to, mono-, di-, and tri-glycerides, preferably di- and tri-glycerides, more preferably triglycerides. For use in the compositions described herein, the glycerides are preferably the mono-, di-, and tri-esters of glycerol and long chain carboxylic acids, such as C₁₀ to C₂₂ carboxylic acids. A variety of these types of materials can be obtained from vegetable and animal fats and oils, such as castor oil, safflower oil, cottonseed oil, corn oil, olive oil, cod liver oil, almond oil, avocado oil, palm oil, sesame oil, lanolin and soybean oil. Synthetic oils include, but are not limited to, triolein and tristearin glyceryl dilaurate.

Other fatty esters suitable for use in the compositions of the present invention are water insoluble synthetic fatty esters. Some preferred synthetic esters conform to the general Formula (IX):

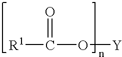

wherein R¹ is a C₇ to C₉ alkyl, alkenyl, hydroxyalkyl or hydroxyalkenyl group, preferably a saturated alkyl group, more preferably a saturated, linear, alkyl group; n is a positive integer having a value from 2 to 4, preferably 3; and Y is an alkyl, alkenyl, hydroxy or carboxy substituted alkyl or alkenyl, having from about 2 to about 20 carbon atoms, preferably from about 3 to about 14 carbon atoms. Other preferred synthetic esters conform to the general Formula (X):

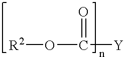

wherein R² is a C₈ to C₁₀ alkyl, alkenyl, hydroxyalkyl or hydroxyalkenyl group; preferably a saturated alkyl group, more preferably a saturated, linear, alkyl group; n and Y are as defined above in Formula (X).

Specific non-limiting examples of suitable synthetic fatty esters for use in the compositions of the present invention include: P-43 (C₈-C₁₀ triester of trimethylolpropane), MCP-684 (tetraester of 3,3 diethanol-1,5 pentadiol), MCP 121 (C₈-C₁₀ diester of adipic acid), all of which are available from Mobil Chemical Company.

d. Additional Conditioning Agents

Also suitable for use in the compositions herein are the conditioning agents described by the Procter & Gamble Company in U.S. Pat. Nos. 5,674,478, and 5,750,122. Also suitable for use herein are those conditioning agents described in U.S. Pat. No. 4,529,586 (Clairol), U.S. Pat. No. 4,507,280 (Clairol), U.S. Pat. No. 4,663,158 (Clairol), U.S. Pat. No. 4,197,865 (L'Oreal), U.S. Pat. No. 4,217, 914 (L'Oreal), U.S. Pat. No. 4,381,919 (L'Oreal), and U.S. Pat. No. 4,422, 853 (L'Oreal).

D. Anti-dandruff Actives

The compositions of the present invention may also contain an anti-dandruff agent. Suitable, non-limiting examples of anti-dandruff particulates include: pyridinethione salts, azoles, selenium sulfide, particulate sulfur, and mixtures thereof. Preferred are pyridinethione salts. Such anti-dandruff particulate should be physically and chemically compatible with the essential components of the composition, and should not otherwise unduly impair product stability, aesthetics or performance.

1. Pyridinethione Salts

Pyridinethione anti-dandruff particulates, especially 1-hydroxy-2-pyridinethione salts, are highly preferred particulate anti-dandruff agents for use in compositions of the present invention. The concentration of pyridinethione anti-dandruff particulate typically ranges from about 0.1% to about 4%, by weight of the composition, preferably from about 0.1% to about 3%, more preferably from about 0.3% to about 2%. Preferred pyridinethione salts include those formed from heavy metals such as zinc, tin, cadmium, magnesium, aluminum and zirconium, preferably zinc, more preferably the zinc salt of 1-hydroxy-2-pyridinethione (known as “zinc pyridinethione” or “ZPT”), more preferably 1-hydroxy-2-pyridinethione salts in platelet particle form, wherein the particles have an average size of up to about 20 μ, preferably up to about 5 μ, more preferably up to about 2.5 μ. Salts formed from other cations, such as sodium, may also be suitable. Pyridinethione anti-dandruff agents are described, for example, in U.S. Pat. No. 2,809,971; U.S. Pat. No. 3,236,733; U.S. Pat. No. 3,753,196; U.S. Pat. No. 3,761,418; U.S. Pat. No. 4,345,080; U.S. Pat. No. 4,323,683; U.S. Pat. No. 4,379,753; and U.S. Pat. No. 4,470,982. It is contemplated that when ZPT is used as the anti-dandruff particulate in the compositions herein, that the growth or re-growth of hair may be stimulated or regulated, or both, or that hair loss may be reduced or inhibited, or that hair may appear thicker or fuller.

2. Other Anti-Microbial Actives

In addition to the anti-dandruff active selected from polyvalent metal salts of pyrithione, the present invention may further comprise one or more anti-fungal or anti-microbial actives in addition to the metal pyrithione salt actives. Suitable anti-microbial actives include coal tar, sulfur, whitfield's ointment, castellani's paint, aluminum chloride, gentian violet, octopirox (piroctone olamine), ciclopirox olamine, undecylenic acid and its metal salts, potassium permanganate, selenium sulphide, sodium thiosulfate, propylene glycol, oil of bitter orange, urea preparations, griseofulvin, 8-Hydroxyquinoline ciloquinol, thiobendazole, thiocarbamates, haloprogin, polyenes, hydroxypyridone, morpholine, benzylamine, allylamines (such as terbinafine), tea tree oil, clove leaf oil, coriander, palmarosa, berberine, thyme red, cinnamon oil, cinnamic aldehyde, citronellic acid, hinokitol, ichthyol pale, Sensiva SC-50, Elestab HP-100, azelaic acid, lyticase, iodopropynyl butylcarbamate (IPBC), isothiazalinones such as octyl isothiazalinone and azoles, and combinations thereof. Preferred anti-microbials include itraconazole, ketoconazole, selenium sulphide and coal tar.

a. Azoles

Azole anti-microbials include imidazoles such as benzimidazole, benzothiazole, bifonazole, butaconazole nitrate, climbazole, clotrimazole, croconazole, eberconazole, econazole, elubiol, fenticonazole, fluconazole, flutimazole, isoconazole, ketoconazole, lanoconazole, metronidazole, miconazole, neticonazole, omoconazole, oxiconazole nitrate, sertaconazole, sulconazole nitrate, tioconazole, thiazole, and triazoles such as terconazole and itraconazole, and combinations thereof. When present in the composition, the azole anti-microbial active is included in an amount from about 0.01% to about 5%, preferably from about 0.1% to about 3%, and more preferably from about 0.3% to about 2%, by weight of the composition. Especially preferred herein is ketoconazole.

b. Selenium Sulfide

Selenium sulfide is a particulate anti-dandruff agent suitable for use in the anti-microbial compositions of the present invention, effective concentrations of which range from about 0.1% to about 4%, by weight of the composition, preferably from about 0.3% to about 2.5%, more preferably from about 0.5% to about 1.5%. Selenium sulfide is generally regarded as a compound having one mole of selenium and two moles of sulfur, although it may also be a cyclic structure that conforms to the general formula SeₓS_{y}, wherein x+y=8. Average particle diameters for the selenium sulfide are typically less than 15 μm, as measured by forward laser light scattering device (e.g. Malvern 3600 instrument), preferably less than 10 μm. Selenium sulfide compounds are described, for example, in U.S. Pat. No. 2,694,668; U.S. Pat. No. 3,152,046; U.S. Pat. No. 4,089,945; and U.S. Pat. No. 4,885,107.

c. Sulfur

Sulfur may also be used as a particulate anti-microbial/anti-dandruff agent in the anti-microbial compositions of the present invention. Effective concentrations of the particulate sulfur are typically from about 1% to about 4%, by weight of the composition, preferably from about 2% to about 4%.

d. Keratolytic Agents

The present invention may further comprise one or more keratolytic agents such as Salicylic Acid.

e. Additional Anti-microbial Actives

Additional anti-microbial actives of the present invention may include extracts of melaleuca (tea tree) and charcoal. The present invention may also comprise combinations of anti-microbial actives. Such combinations may include octopirox and zinc pyrithione combinations, pine tar and sulfur combinations, salicylic acid and zinc pyrithione combinations, octopirox and climbasole combinations, and salicylic acid and octopirox combinations, and mixtures thereof.

E. Humectant

The compositions of the present invention may contain a humectant. The humectants herein are selected from the group consisting of polyhydric alcohols, water soluble alkoxylated nonionic polymers, and mixtures thereof. The humectants, when used herein, are preferably used at levels of from about 0.1% to about 20%, more preferably from about 0.5% to about 5%.

Polyhydric alcohols useful herein include glycerin, sorbitol, propylene glycol, butylene glycol, hexylene glycol, ethoxylated glucose, 1, 2-hexane diol, hexanetriol, dipropylene glycol, erythritol, trehalose, diglycerin, xylitol, maltitol, maltose, glucose, fructose, sodium chondroitin sulfate, sodium hyaluronate, sodium adenosine phosphate, sodium lactate, pyrrolidone carbonate, glucosamine, cyclodextrin, and mixtures thereof.

Water soluble alkoxylated nonionic polymers useful herein include polyethylene glycols and polypropylene glycols having a molecular weight of up to about 1000 such as those with CTFA names PEG-200, PEG-400, PEG-600, PEG-1000, and mixtures thereof.

F. Suspending Agent

The compositions of the present invention may further comprise a suspending agent at concentrations effective for suspending water-insoluble material in dispersed form in the compositions or for modifying the viscosity of the composition. Such concentrations range from about 0.1% to about 10%, preferably from about 0.3% to about 5.0%.

Suspending agents useful herein include anionic polymers and nonionic polymers. Useful herein are vinyl polymers such as cross linked acrylic acid polymers with the CTFA name Carbomer, cellulose derivatives and modified cellulose polymers such as methyl cellulose, ethyl cellulose, hydroxyethyl cellulose, hydroxypropyl methyl cellulose, nitro cellulose, sodium cellulose sulfate, sodium carboxymethyl cellulose, crystalline cellulose, cellulose powder, polyvinylpyrrolidone, polyvinyl alcohol, guar gum, hydroxypropyl guar gum, xanthan gum, arabia gum, tragacanth, galactan, carob gum, guar gum, karaya gum, carragheenin, pectin, agar, quince seed (Cydonia oblonga Mill), starch (rice, corn, potato, wheat), algae colloids (algae extract), microbiological polymers such as dextran, succinoglucan, pulleran, starch-based polymers such as carboxymethyl starch, methylhydroxypropyl starch, alginic acid-based polymers such as sodium alginate, alginic acid propylene glycol esters, acrylate polymers such as sodium polyacrylate, polyethylacrylate, polyacrylamide, polyethyleneimine, and inorganic water soluble material such as bentonite, aluminum magnesium silicate, laponite, hectonite, and anhydrous silicic acid.

Commercially available viscosity modifiers highly useful herein include Carbomers with tradenames Carbopol 934, Carbopol 940, Carbopol 950, Carbopol 980, and Carbopol 981, all available from B. F. Goodrich Company, acrylates/steareth-20 methacrylate copolymer with tradename ACRYSOL 22 available from Rohm and Hass, nonoxynyl hydroxyethylcellulose with tradename AMERCELL POLYMER HM-1500 available from Amerchol, methylcellulose with tradename BENECEL, hydroxyethyl cellulose with tradename NATROSOL, hydroxypropyl cellulose with tradename KLUCEL, cetyl hydroxyethyl cellulose with tradename POLYSURF 67, all supplied by Hercules, ethylene oxide and/or propylene oxide based polymers with tradenames CARBOWAX PEGs, POLYOX WASRs, and UCON FLUIDS, all supplied by Amerchol.

Other optional suspending agents include crystalline suspending agents which can be categorized as acyl derivatives, long chain amine oxides, and mixtures thereof. These suspending agents are described in U.S. Pat. No. 4,741,855. These preferred suspending agents include ethylene glycol esters of fatty acids preferably having from about 16 to about 22 carbon atoms. More preferred are the ethylene glycol stearates, both mono and distearate, but particularly the distearate containing less than about 7% of the mono stearate. Other suitable suspending agents include alkanol amides of fatty acids, preferably having from about 16 to about 22 carbon atoms, more preferably about 16 to 18 carbon atoms, preferred examples of which include stearic monoethanolamide, stearic diethanolamide, stearic monoisopropanolamide and stearic monoethanolamide stearate. Other long chain acyl derivatives include long chain esters of long chain fatty acids (e.g., stearyl stearate, cetyl palmitate, etc.); long chain esters of long chain alkanol amides (e.g., stearamide diethanolamide distearate, stearamide monoethanolamide stearate); and glyceryl esters (e.g., glyceryl distearate, trihydroxystearin, tribehenin) a commercial example of which is Thixin R available from Rheox, Inc. Long chain acyl derivatives, ethylene glycol esters of long chain carboxylic acids, long chain amine oxides, and alkanol amides of long chain carboxylic acids in addition to the preferred materials listed above may be used as suspending agents.

Other long chain acyl derivatives suitable for use as suspending agents include N,N-dihydrocarbyl amido benzoic acid and soluble salts thereof (e.g., Na, K), particularly N,N-di(hydrogenated) C.sub. 16, C.sub. 18 and tallow amido benzoic acid species of this family, which are commercially available from Stepan Company (Northfield, Ill., USA).

Examples of suitable long chain amine oxides for use as suspending agents include alkyl dimethyl amine oxides, e.g., stearyl dimethyl amine oxide. Other suitable suspending agents include primary amines having a fatty alkyl moiety having at least about 16 carbon atoms, examples of which include palmitamine or stearamine, and secondary amines having two fatty alkyl moieties each having at least about 12 carbon atoms, examples of which include dipalmitoylamine or di(hydrogenated tallow)amine. Still other suitable suspending agents include di(hydrogenated tallow)phthalic acid amide, and crosslinked maleic anhydride-methyl vinyl ether copolymer.

G. Other Additional Components

The compositions of the present invention may contain also vitamins and amino acids such as: water soluble vitamins such as vitamin B1, B2, B6, B12, C, pantothenic acid, pantothenyl ethyl ether, panthenol, biotin, and their derivatives, water soluble amino acids such as asparagine, alanin, indole, glutamic acid and their salts, water insoluble vitamins such as vitamin A, D, E, and their derivatives, water insoluble amino acids such as tyrosine, tryptamine, and their salts.

The compositions of the present invention may also contain pigment materials such as inorganic, nitroso, monoazo, disazo, carotenoid, triphenyl methane, triaryl methane, xanthene, quinoline, oxazine, azine, anthraquinone, indigoid, thionindigoid, quinacridone, phthalocianine, botanical, natural colors, including: water soluble components such as those having C. I. Names.

The compositions of the present invention may also contain antimicrobial agents which are useful as cosmetic biocides and antidandruff agents including: water soluble components such as piroctone olamine, water insoluble components such as 3,4,4′-trichlorocarbanilide (trichlosan), triclocarban and zinc pyrithione.

The compositions of the present invention may also contain chelating agents.

VI. Method of Making an Aminosilicone and NAFS Conditioning Shampoo

Another embodiment of the present invention relates to a method of making a conditioning shampoo composition, comprising mixing together: a) a previously formed blend of aminosilicone and NAFS, b) a surfactant, and c) water; wherein the aminosilicone has less than about 0.5% nitrogen by weight of the aminosilicone and the NAFS has a viscosity of at least about 10,000 cs. Other embodiments of aminosilicone and NAFS set forth above, are also useful in such a method of making a conditioning shampoo composition.

More specifically, the aminosilicone and NAFS are first blended/mixed together before being emulsified either into the shampoo or in a premix. The blend is then emulsified either directly into the shampoo, or in a premix that is then added to the shampoo. This method results in emulsified droplets in the shampoo composition that each contain the desired ratio of amino to non-amino functionalized silicones. This has a very different result than in separately emulsified amino and non-amino functionalized silicone droplets. In the emulsified blend the aminosilicone helps the deposition of the non-amino functionalized silicone component. In the blend, the aminosilicone is surface active and thus concentrates at the surface of the blended droplet thus lowering interfacial tensions and aiding spreading and deposition on the hair.

The following examples further describe and demonstrate the preferred embodiments within the scope of the present invention. The examples are given solely for the purpose of illustration, and are not to be construed as limitations of the present invention since many variations thereof are possible without departing from its scope.

### EXAMPLE 1

Example 1 demonstrates the surprising discovery that aminosilicones below a certain percent nitrogen range will provide a superior level of reduced friction on treated hair.

Table 1 shows the relationship between friction and percent nitrogen for amino functionalized silicone.

TABLE 1

| | Amine | | | Coef. of |
|---|---|---|---|---|
| Position | Type | VISC | % N | Friction |
| None | — | 350,000 | 0 | 0.390 |
| Term | AP | 412,000 | 0.022 | 0.298 |
| Term | AP | 312,000 | 0.026 | 0.290 |
| Term | AP | 111,875 | 0.032 | 0.282 |
| Term | AP | 87,000 | 0.035 | 0.300 |
| Term | AP | 55,950 | 0.038 | 0.277 |
| Term | AP | 31,000 | 0.051 | 0.282 |
| Term | AP | 10,450 | 0.053 | 0.311 |
| Term | AP | 22,980 | 0.060 | 0.294 |
| Term | AP | 12,338 | 0.070 | 0.298 |
| Graft | AEAP | 46,200 | 0.074 | 0.322 |
| Term | AP | 8,391 | 0.076 | 0.292 |
| Term | AP | 7,029 | 0.077 | 0.304 |
| Term | AP | 5,113 | 0.087 | 0.282 |
| Term | AEAP | 24,160 | 0.113 | 0.341 |
| Term | AP | 2,038 | 0.126 | 0.307 |
| Graft | AP | 191,800 | 0.164 | 0.426 |
| Graft | AEAP | 1,000,000+ | 0.176 | 0.401 |
| Graft | AEAP | 1,000,000+ | 0.182 | 0.386 |
| Graft | AP | 53,400 | 0.182 | 0.390 |
| Graft | AEAP | 78,400 | 0.196 | 0.338 |
| Graft | AEAP | 558,000 | 0.211 | 0.354 |
| Graft | AEAP | 1,000,000+ | 0.504 | 0.490 |
| Graft | AEAP | 98,500 | 0.616 | 0.472 |
| Graft | AP | 1,000,000+ | 0.616 | 0.520 |
| Graft | AP | 143,500 | 0.637 | 0.509 |

For this study the silicone is dissolved in a volatile solvent, hexamethyl disiloxane (MM), and applied to hair (20 gram flat switch) or 2 gram paper strip (3 inches by 9 inches (7.62 cm×22.86 cm)) at a level of 1,000 ppm of silicone to hair/paper weight. The solvent is allowed to evaporate and the hair/paper is allowed to equilibrate in a 50% relative humidity overnight. The friction of the coated hair/paper is then measured using an Instron model 5542 (Instron, Inc.; Canton, Mass., USA) to measure the force to drag a weighted sled (100 gms of weight) along the hair/paper in the with-cuticle direction.

### EXAMPLES 2-6

Examples 2-6 illustrate non-limiting hair conditioning shampoo composition embodiments of the present invention. These compositions are prepared by conventional formulation and mixing methods, an example of which is set forth, below. All exemplified amounts are listed as weight percents and exclude minor materials such as diluents, filler, and the like, unless otherwise specified. The listed formulations, therefore, comprise the listed components and any minor materials associated with such components.

The compositions illustrated in Examples 2-6 are prepared in the following manner.

For each of the compositions, 6-9% of ammonium laureth-3 sulfate, P43 oil, PureSyn6 oil, cationic polymers, 0-1.5% ammonium xylene sulfonate, and 0-5% water is added to a jacketed mix tank and heated to about 74° C. with agitation to form a solution. Citric acid, sodium citrate, sodium benzoate, disodium EDTA, cocamide MEA and 0.6-0.9% cetyl alcohol, are added to the tank and allowed to disperse. Ethylene glycol distearate (EGDS) is then added to the mixing vessel, and melted. After the EGDS is well dispersed (after about 10 minutes) preservative is added and mixed into the surfactant solution. This mixture is passed through a mill and heat exchanger where it is cooled to about 35° C. and collected in a finishing tank. As a result of this cooling step, the ethylene glycol distearate crystallize to form a crystalline network in the product.

The silicone is pre-emulsified to the desired particle size by mixing with surfactants/polymers selected from laureth sulfate, Plantarem2000® and Structure Plus®.

The remainder of the surfactants, perfume, pre-emulsified silicone, dimethicone, sodium chloride or ammonium xylene sulfonate for viscosity adjustment and the remainder of the water are added to the finishing tank with ample agitation to ensure a homogeneous mixture.

Preferred viscosities range from about 5000 to about 9000 centipoise at 27° C. (as measured by a Wells-Brookfield model RVTDCP viscometer using a CP-41 cone and plate at 2/s at 3 minutes).

For those compositions containing an aminosilicone and NAFS (e.g., a polydimethylsiloxane such as dimethicone), the aminosilicone and NAFS are first blended/mixed together before being emulsified either into the shampoo or in a premix. The blend is then emulsified either directly into the shampoo, or in a premix that is then added to the shampoo.

| | Example No. | | | | | |
|---|---|---|---|---|---|---|
| Component | 2 | 3 | 4 | 5 | 6 | 7 |
| Water-USP Purified & | Q.S. to | Q.S. to | Q.S. to | Q.S. to | Q.S. to | Q.S. to |
| Minors | 100 | 100 | 100 | 100 | 100 | 100 |
| Ammonium Laureth Sulfate | 10 | 11.67 | 10 | 10 | 10 | 10 |
| Ammonium Lauryl Sulfate | 6 | 2.33 | 4 | 6 | 6 | 6 |
| Cocaminopropionic acid | — | 2 | 2 | — | — | — |
| Puresyn 6 (1-decene | 0.4 | 0.25 | 0.25 | 0.4 | 0.4 | — |
| homopolymer) | | | | | | |
| Trimethylolpropane | 0.1 | — | — | 0.1 | 0.1 | — |
| Tricaprylate/ | | | | | | |
| Tricaprate | | | | | | |
| Cocamide MEA | 0.8 | 0.8 | 0.8 | 0.8 | 0.8 | 0.8 |
| Citric Acid | 0.04 | 0.04 | 0.04 | 0.04 | 0.04 | 0.04 |
| Sodium Citrate Dihydrate | 0.4 | 0.4 | 0.4 | 0.4 | 0.4 | 0.4 |
| Disodium EDTA | 0.1 | 0.1 | 0.1 | 0.1 | 0.1 | 0.1 |
| Kathon | 0.0005 | 0.0005 | 0.0005 | 0.0005 | 0.0005 | 0.0005 |
| Sodium Benzoate | 0.25 | 0.25 | 0.25 | 0.25 | 0.25 | 0.25 |
| Disodium EDTA | 0.1274 | 0.1274 | 0.1274 | 0.1274 | 0.1274 | 0.1274 |
| Cetyl Alcohol | 0.9 | 0.6 | 0.6 | 0.9 | 0.9 | 0.9 |
| Ethylene Glycol Distearate | 1.5 | 1.5 | 1.5 | 1.5 | 1.5 | 1.5 |
| Polyox PEG7M | — | — | — | — | 0.1 | 0.1 |
| Polyquaternium-10 (KG30M) | — | 0.5 | 0.5 | 0.5 | — | — |
| Polyquaternium-10 (LR30M) | — | — | — | — | 0.5 | — |
| Guar | 0.5 | — | — | — | — | — |
| Hydroxypropyltrimonium | | | | | | |
| Chloride¹ | | | | | | |
| Dimethicone (Viscasil 330M) | 2.23 | — | 1.9 | — | 2.0 | — |
| Aminosilicone² | 0.12 | 2 | — | 1 | 0.22 | — |
| Aminosilicone³ | — | — | 0.1 | — | — | — |
| Aminosilicone⁴ | — | — | — | — | — | 2.4 |
| Structure Plus⁵ | — | — | — | — | — | .08 |
| Plantarem 2000 | — | 0.125 | 0.055 | 0.025 | — | .08 |
| Perfume | 0.7 | 0.7 | 0.7 | 0.7 | 0.7 | 0.7 |
| Sodium Chloride | 0-3 | 0-3 | 0-3 | 0-3 | 0-3 | 0-3 |
| Ammonium Xylene Sulfonate | 0-3 | 0-3 | 0-3 | 0-3 | 0-3 | 0-3 |
| Particle Size | L | S | S | S | L | L |

¹Guar having a molecular weight of about 400,000, and having a charge density of about 2.10 meq/g, available from Aqualon.
²Aminosilicone from GE with terminal aminopropyl substitution, viscosity ˜8000 cps, D˜490, M' = 2.
³Aminosilicone from GE with graft aminoethylaminopropyl substitution, viscosity ˜20,000 cps, D˜600, D'˜2.
⁴Aminosilicone from GE with terminal aminopropyl substitution, viscosity ˜350,000, D˜1600, M' = 2.
⁵National Starch (20%) acrylates aminoacrylates copolymer .08%
L = 5 to 50 μ particle size, S = <5μ particle size

It is understood that the examples and embodiments described herein are for illustrative purposes only and that various modifications or changes in light thereof will be suggested to one skilled in the art without departing from the scope of the present invention.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A conditioning shampoo composition comprising:
a) a detersive surfactant
b) an aminosilicone having a viscosity of from about 1,000 cs to about 1,000,000 cs, and less than about 0.5% nitrogen by weight of the aminosilicone and;
c) an aqueous carrier.

2. The composition of claim 1, wherein said aminosilicone is a terminal aminosilicone with a viscosity of from about 50,000 cs to about 1,000,000 cs.

3. The composition of claim 1, wherein said aminosilicone is a grafted aminosilicone with a viscosity of from about 1,000 cs to about 50,000 cs.

4. The composition of claim 1, wherein said aminosilicone has a viscosity of from about 5,000 cs to about 600,000 cs.

5. The composition of claim 1, wherein said aminosilicone has a viscosity of from about 10,000 cs to about 400,000 cs.

6. The composition of claim 1, wherein said aminosilicone has a viscosity of from about 50,000 cs to about 100,000 cs.

7. The composition of claim 1, wherein the aminosilcone has an average particle size of from about 5 microns to about 50 microns.

8. The composition of claim 1, wherein the aminosilicone has an average particle size of less than about 5 microns.

9. The composition of claim 8, further comprising a deposition aid.

10. The composition of claim 9, wherein the deposition aid is a cationic polymer.

11. The composition of claim 1, wherein the aminosilicone has less than about 0.2% nitrogen by weight of the aminosilicone.

12. A conditioning shampoo composition comprising:
a) a detersive surfactant;
b) an aminosilicone having less than about 0.5% nitrogen by weight of the aminosilicone;
c) a non-amino-functionalized silicone having a viscosity of at least about 10,000 cs and
d) an aqueous carrier.

13. The composition of claim 12, wherein the aminosilicone and non-amino functionalized silicone are in the form of emulsion drops containing a blend of the aminosilicone and non-amino-functionalized silicone.

14. The composition of claim 13, wherein the emulsion drops have an average particle size of from about 5 microns to about 50 microns.

15. The composition of claim 13, wherein the emulsion drops have an average particle size of less than about 5 microns.

16. The composition of claim 15, further comprising a deposition aid.

17. The composition of claim 16, wherein the deposition aid is a cationic polymer.

18. The composition of claim 12, wherein the aminosilicone has less than about 0.2% nitrogen by weight of the aminosilicone.

19. The composition of claim 12, wherein the ratio of the aminosilicone to the non-amino functionalized silicone is from about 1:5 to about 0.1:99.9.

20. A method of making a conditioning shampoo composition comprising mixing together:
a) a previously formed blend of aminosilicone and non-amino-functionalized silicone, wherein the aminosilicone has less than about 0.5% nitrogen by weight of the aminosilicone and the non-amino-functionalized silicone has a viscosity of at least about 10,000 cs;
b) a detersive surfactant, and
c) an aqueous carrier.
